(19) United States
(12) Patent Application Publication
(10) Pub. No.: US 2014/0080083 A1
(43) Pub. Date: Mar. 20, 2014

(54) FUNCTIONAL DENTOFACIAL ORTHOPEDIC APPARATUS

(71) Applicant: Rodrigue Mathieu, Montpellier (FR)

(72) Inventor: Rodrigue Mathieu, Montpellier (FR)

(21) Appl. No.: 14/007,885

(22) PCT Filed: Mar. 13, 2012
(86) PCT No.: PCT/FR2012/050523

§ 371 Date: Nov. 26, 2013

(30) Foreign Application Priority Data
Apr. 1, 2011 (FR) 11 00983

(51) Int. Cl. A61F 5/56; A61F 5/58; A63B 71/08; A61C 5/14; A61C 7/08

(52) U.S. Cl. CPC A61F 5/566; A61C 5/14; A61C 7/08; A63B 71/085; A61F 5/58
USPC 433/6; 128/860; 128/848

(57) ABSTRACT

The invention relates to a functional dentofacial orthopedic apparatus (1) made of a flexible material and comprising two grooves, the general outer shape of each of which is a flared U when viewed horizontally, the two grooves being separated by a substantially horizontal partition (6). Each groove defines an alveolar canal (4, 5) encompassing the superior and inferior alveolodental arches, respectively, said canal (4, 5) consisting of a labial bar (2) and a lingual plate (3), as well as the horizontal partition (6), the inner surface of each canal being smooth. According to the invention, the apparatus includes: a continuous angled lingual ramp (10) which is intended for supporting and guiding the tongue toward the palate and the upper portion of which consists of a reinforced inner raised portion (9), the general shape of which is flared U and which extends over the inner circumference of the grooves. The cross-section of said raised portion gradually increases from a minimum value at the end of said raised portion to a maximum value in the central portion of the latter.

### FIELD OF THE INVENTION

The present invention relates to a functional dentofacial orthopedic apparatus for correcting functional neurovegetative function disorders, particularly nasal ventilation, chewing, swallowing and phonation disorders to optimize occlusion and enable bone development and repositioning of the teeth in a functional and cosmetic aim.

### STATE OF THE RELATED ART

To correct the position of teeth in patients, functional orthopedics generally uses removable corrective apparatuses. These corrective apparatuses are of the “orthodontic positioner” type or “splint activator” type, they are generally made of a flexible material, and, when inserted into the mouth, they exert pressures on the incorrectly positioned teeth to correct the dental bones and the relationship between the two jaws.

A first type of functional orthopedic apparatuses consists of “orthodontic positioners”. These apparatuses are produced individually for each patient. Such an apparatus is described in the document EP 0 315 777, has a general U shape and comprises, on either side of a central horizontal wall, notches for receiving the teeth of the upper and lower arches. The apparatus described in this document also comprises means for guiding the tongue comprising a pointed projection formed on the lingual side of the lower median part of the apparatus and a hollow area situated above the projection for receiving the end of the tongue. The primary drawback of the apparatus is that of having to position the teeth in precise slots in the shape of the teeth, with movements of teeth inside the positioned not being permitted. Moreover, the protuberances from the median part of the apparatus are intended to discourage an incorrect position of the tongue, but they merely orient the tip of the tongue in relation to a median line of the apparatus in order to prevent same from adopting an excessively advanced or excessively low position.

Splint activators are apparatuses of a standard shape and size. The document EP 0 925 039 filed by the applicant describes such a type of appliance comprising two grooves having a flared U shape, which are separated by a horizontal partition and wherein the inner surface is smooth. The walls having a smooth inner surface of the grooves are defined between a labial bar and a lingual and palate bar and the horizontal partition. More specifically, according to this document, the portion of the groove intended to receive the lower incisors is in the shape of a straight canal opening at the ends thereof into two alveolar canals intended to receive a lower canine, the premolars and at least one lower molar.

Furthermore, the document FR 2 641 964 describes a further example of a splint activator comprising grooves each having a semi-elliptical shape, the inner surfaces thereof having smooth walls, each groove having a rounded canal for receiving the incisors.

A splint activator is generally made of a food-grade flexible material, based on natural or synthetic rubber. When it is placed in a patient's oral cavity, the splint activator sets an ideal position of the teeth while stimulating the muscles of the face and mouth during various movements of the natural dentomaxillofacial functions. The muscles are thus strengthened according to the correction applied by the activator. While enabling correction of the position of the teeth, enhanced occlusion and function of the muscles of the face and mouth, however, it has been observed that these apparatuses displayed limitations when infraclusions and gaps created in the vertical direction, between the upper and lower incisors, appear during treatment or if they existed prior to treatment. This is essentially due to an incorrect position of the tongue, combined with atypical swallowing.

To attempt to remedy this problem, the document FR 2 867 058 proposed a lingual positioning apparatus equipped with a concave lingual ramp allowing the tongue to bear thereon and move up to the palate, and a tab positioned directly above the lingual ramp forming a second bearing point for the tongue. The lingual ramp is held in the central part thereof by a convex shaped supporting member. Such an apparatus indeed enables superior orientation and stimulation of the tongue than previous apparatuses, but merely in the median part of the apparatus.

The aim of the present invention is that of remedying these drawbacks at least in part and proposing a dentofacial orthopedic apparatus suitable for correct orientation of the tongue, from the insertion of the apparatus into a patient's oral cavity, while ensuring superior muscle tone in the mouth and teeth of a patient using the apparatus.

A further aim of the invention is that of proposing a dentofacial orthopedic apparatus providing superior stimulant of the muscles of the face and mouth for enhanced treatment efficacy, while minimizing the risk of recurrence or appearance of further malposition of the teeth.

A further aim of the invention is that of proposing a dentofacial orthopedic apparatus which is comfortable to use, while providing effective and reliable treatment.

A further aim of the invention is that of proposing a dentofacial orthopedic appliance having a standard size which can be readily adapted to the patient to be treated, while being suitable for economical mass production.

### SUBJECT MATTER OF THE INVENTION

The aims of the invention are achieved with a functional dentofacial orthopedic apparatus made of a flexible material and comprising two grooves, the general outer shape of each of which is a flared U when viewed horizontally, the two grooves being separated by a substantially horizontal partition, each groove defines an alveolar canal encompassing the superior and inferior alveolodental arches, said canal consisting of a labial bar and a lingual bar, as well as the horizontal partition, the inner surface of each canal being smooth, in that it comprises:
a continuous angled lingual ramp for supporting and guiding the tongue toward the palate formed on the upper portion,
a reinforced inner raised portion, the general shape of which is a flared U and which extends over the inner periphery of the grooves and the cross-section of which gradually increases from a minimum value at the end of said raised portion to a maximum value in the central portion thereof.

The apparatus is made of a food-grade or biocompatible flexible material, so that, when worn in the mouth and chewing exercises are carried out, this appliance can have elastic properties, without injuring the mucosa. The apparatus has a double-grooved shape encompassing both arches simultaneously. Due to the elastic properties thereof, the apparatus according to the invention stimulates the muscles of the face and mouth during chewing exercises. The apparatus always returns to the primary shape thereof which is that of ideal arches positioned in a normal or corrected relationship.

The apparatuses according to the invention are specifically for children and adults, have standardized shapes and sizes according to the patient's age or the defect to be treated by rehabilitating the neurovegetative functions.

According to the invention, the dentofacial orthopedic apparatus comprises a continuous angled lingual ramp for orienting the tongue toward the palate, the ramp being formed on the upper portion of a reinforced inner raised portion, the general shape of which is a flared U and which extends over all or most of the inner periphery of the grooves. Such a ramp forms a continuous bearing surface for the tongue, on the periphery thereof. The reinforced inner raised portion thus has substantially the same shape as the grooves and it is embodied in the form of a continuous rim, the contour of which, as viewed in the horizontal plane, is defined, from the alveolar canal, by the lingual bar, and internally by an inner bar for inserting the tongue. This reinforced inner raised portion advantageously has a cross-section which increases from a minimum value at one of the ends of the inner raised portion (or at the end of a first lateral branch of the U) to a maximum value attained in the central portion thereof (or at the base of the U, or at the center of said base). The apparatus according to the invention is symmetric, meaning that the value of the cross-section of the reinforced inner raised portion attaining the maximum value thereof in the central portion thereof subsequently decreases from the central portion to the end of the opposite lateral branch. In this way, the apparatus exhibits increased strength in the central portion thereof due to the presence of an inner bar of substantially constant thickness forming a reinforced inner raised portion. The term central portion refers to the area located in the middle of the alveolar canal for receiving the incisors, or the area situated substantially in the vicinity of the median axis of the apparatus or, in some alternative embodiments of the invention, in the area of the alveolar canal starting at the canines and extending therebetween. Such an inner raised portion makes it possible, due to the large size thereof, to form a supporting member for the tongue, the bearing surface enabling progressive guiding of the tongue, at the lateral sides thereof, and a larger bearing surface for the central portion of the tongue, while offering, due to the reinforced strength thereof, superior tone during stimulations of the tongue, but also of the muscles of the face and mouth during chewing or training exercises.

Indeed, due to the large size of the inner raised portion and the presence of the angled ramp on the periphery of the reinforced inner raised portion, the tongue is required to bear on the angled ramp thus orienting same toward the palate, from the insertion of the apparatus into the mouth. This provides superior nasal ventilation and satisfactory swallowing. Furthermore, when bearing on the ramp of the inner raised portion, the tongue produces stimulations in contact with the inner raised supporting portion by pushing toward the alveolar arches, on the periphery of the reinforced inner raised portion. These stimulations are added to those of the muscles of the mouth and face produced during chewing exercises performed with the apparatus, stimulating and developing muscle tone. All the muscles involved (depressor, levator and perioral muscles) thus gradually adapt to an optimized physiological function, suitable for maintaining the occlusal-articular equilibrium obtained using the apparatus.

In this way, the functional dentofacial orthopedic apparatus according to the invention is suitable for aligning teeth, holding same in the corrected position, while ensuring satisfactory neurovegetative functions, particularly by restoring satisfactory chewing, satisfactory breathing through the nose, satisfactory swallowing with correct positioning of the tongue, phonation, for satisfactory functional and cosmetic results.

Advantageously, the depth of each alveolar canal is at least equal to the height of the teeth encompassed therein, to the neck thereof.

The depth of the alveolar canal is thus sufficient to enable the apparatus to encompass the teeth of the superior arch and the inferior arch from the sharp edge thereof at least to the neck thereof when wearing in the mouth with the teeth closed. This enables satisfactory transmission of the stimulations and excitations from the contact with the teeth for superior efficacy of the apparatus.

Preferably, said angled lingual ramp comprises a substantially plane central slope connected to two substantially plane adjacent lateral slopes.

Such a three-sloped angled ramp makes it possible to orient the tongue correctly toward the palate, from the insertion of the apparatus into the mouth, guiding the tongue not only in the central portion thereof, but also along the lateral portions thereof. Such a ramp comprising substantially plane surfaces provides a larger contact area of the tongue for satisfactory comfort thereof when bearing on the ramp. The angle of the lateral slopes is substantially equal to, or slightly greater than, that of the central slope.

Advantageously, said reinforced inner raised portion has a greater thickness in the area of the alveolar canal of the superior arch and the inferior arch intended to receive the incisors and the canines.

Such an apparatus has an area of reinforced transverse rigidity for providing a greater transverse action and enabling the teeth to be positioned in the area of the alveolar canal of the superior arch and the inferior arch intended to receive the incisors and the canines, while inciting the tongue to be positioned rapidly, in contact with wider bearing surfaces.

Preferably, the apparatus is produced from a food-grade silicone base. This material should not comprise toxic catalyst residues; a silicone comprising traces of platinum is suitable for this purpose. Such a material is generally transparent, the composition thereof may however include a dye base based on biocompatible dyes (typically 1 to 4% by mass). Biocompatible aromas may also be added into the composition of the material for more pleasant use of the apparatus in the mouth.

It was observed during laboratory tests performed that a material based on food-grade silicone particularly suitable for producing the apparatus according to the invention should exhibit satisfactory properties in respect of flexibility and comfort when wearing in the mouth, while having a high elasticity. Furthermore, such a material should have a very high endurance and satisfactory resistance to friction, providing satisfactory treatment efficacy and, at the same time, a longer service life of the apparatus.

Advantageously, said silicone has a hardness between 50 and 60 Shore (as per ASTM D2240), and preferably between 53 and 57 Shore. It is preferred to produce the apparatus according to the invention from a silicone-based material having a compression set between 1.9% and 2.9%, and preferably between 2.2% and 2.6%, measured 30 minutes after the release of the sample from 25% compression for 70 hours. The breaking strength thereof is preferably between 8 and 12 MPa (as per ASTM D882). The elongation at break thereof (as per ASTM D412) is preferably between 820 and 1030% and more preferentially between 875 and 975%. The tear strength thereof is preferably between 46 and 60 kN/m (as per ASTM D624B), and more preferentially between 50 and 56 kN/m. An optimum material for producing apparatuses according to the invention exhibits all of these mechanical characteristics.

In one alternative embodiment of the invention, the portion of the canal intended to receive the lower or upper incisors is in the shape of a straight canal, said canal opens, at both ends thereof, respectively into alveolar canals each intended to receive a lower canine, two lower premolars or a child's two corresponding milk molars and, optionally, at least one lower molar.

Such an apparatus comprising a straight or rectilinear alveolar canal for receiving the incisors makes it possible to create superior transverse expansion of the upper and lower alveolar arches, more effective correction of the shape of the bones in the mouth and corresponding malpositions of the teeth, while preventing vestibulation of the upper and lower incisors.

In a further alternative embodiment of the invention, the portion of the canal intended to receive the lower or upper incisors has a rounded canal shape, said canal opens, at both ends thereof, respectively into alveolar canals each intended to receive one lower canine, two lower premolars or a child's two corresponding milk molars and, optionally, at least one lower molar.

Such an apparatus comprising a rounded alveolar canal for receiving incisors is suitable for making the necessary correction of the defects in the shape of the bones in the mouth and the corresponding dental malpositions, while applying less force, in a transverse direction, on the teeth, and thus being more comfortable when used in the mouth.

The aim of the invention is also achieved with a range of functional dentofacial orthopedic apparatuses, in that it comprises at least two apparatuses according to the invention chosen from: a transverse expansion apparatus, a conformer, an advancement apparatus, a retraction apparatus, a sports training apparatus or an anti-snoring apparatus.

Such a range of apparatuses according to the invention meets expectations in respect of dentofacial orthodontics as the apparatuses in the range succeed in correcting the main defects and nasal ventilation, chewing, swallowing, phonation, occlusion and bone shape disorders, or may be used as a mouth guard during training or sports activities or help prevent snoring and improve breathing when sleeping. Each apparatus in the range may be produced in a plurality of sizes according to the patient's age and morphology.

### DESCRIPTION OF THE FIGURES

FIGS. 1a to 1f illustrate a first example of an embodiment of the apparatus according to the invention where FIG. 1a illustrates a top view, FIG. 1b illustrates a bottom view, FIG. 1c a front view and FIGS. 1d, 1e and 1f sectional views produced with the planes A-A, B-B and C-C in FIGS. 1a and 1b;

FIGS. 2a to 2f illustrate a second example of an embodiment of the apparatus according to the invention where FIG. 2a illustrates a top view, FIG. 2b illustrates a bottom view, FIG. 2c a front view and FIGS. 2d, 2e and 2f sectional views produced with the planes A-A, B-B and C-C in FIGS. 2a and 2b;

FIGS. 3a to 3f illustrate a third example of an embodiment of the apparatus according to the invention where FIG. 3a illustrates a top view, FIG. 3b illustrates a bottom view, FIG. 3c a front view and FIGS. 3d, 3e and 3f sectional views produced with the planes A-A, B-B and C-C in FIGS. 3a and 3b;

FIGS. 4a to 4f illustrate a fourth example of an embodiment of the apparatus according to the invention where FIG. 4a illustrates a top view, FIG. 4b illustrates a bottom view, FIG. 4c a front view and FIGS. 4d, 4e and 4f sectional views produced with the planes A-A, B-B and C-C in FIGS. 4a and 4b;

FIGS. 5a to 5f illustrate a fifth example of an embodiment of the apparatus according to the invention where FIG. 5a illustrates a top view, FIG. 5b illustrates a bottom view, FIG. 5c a front view and FIGS. 5d, 5e and 5f sectional views produced with the planes A-A, B-B and C-C in FIGS. 5a and 5b;

FIGS. 6a to 6f illustrate a sixth example of an embodiment of the apparatus according to the invention where FIG. 6a illustrates a top view, FIG. 6b illustrates a bottom view, FIG. 6c a front view and FIGS. 6d, 6e and 6f sectional views produced with the planes A-A, B-B and C-C in FIGS. 6a and 6b;

FIGS. 7a to 7f illustrate a seventh example of an embodiment of the apparatus according to the invention where FIG. 7a illustrates a top view, FIG. 7b illustrates a bottom view, FIG. 7c a front view and FIGS. 7d, 7e and 7f sectional views produced with the planes A-A, B-B and C-C in FIGS. 7a and 7b.

List of references:

| | |
|---|---|
| 1 | Functional dentofacial orthodontic |
| | apparatus |
| 2 | Labial bar |
| 3 | Lingual bar |
| 4 | Upper alveolar canal |
| 5 | Lower alveolar canal |
| 6 | Horizontal partition |
| 7 | Upper notch |
| 8 | Lower notch |
| 9 | Reinforced raised portion |
| 10 | Lingual ramp |
| 11 | Central portion |
| 12 | Lateral branch |
| 13 | Central slope |
| 14 | Lateral slope |
| 15 | Straight upper canal |
| 16 | Straight lower canal |
| 17 | Rounded upper canal |
| 18 | Rounded lower canal |

### DETAILED DESCRIPTION OF THE INVENTION

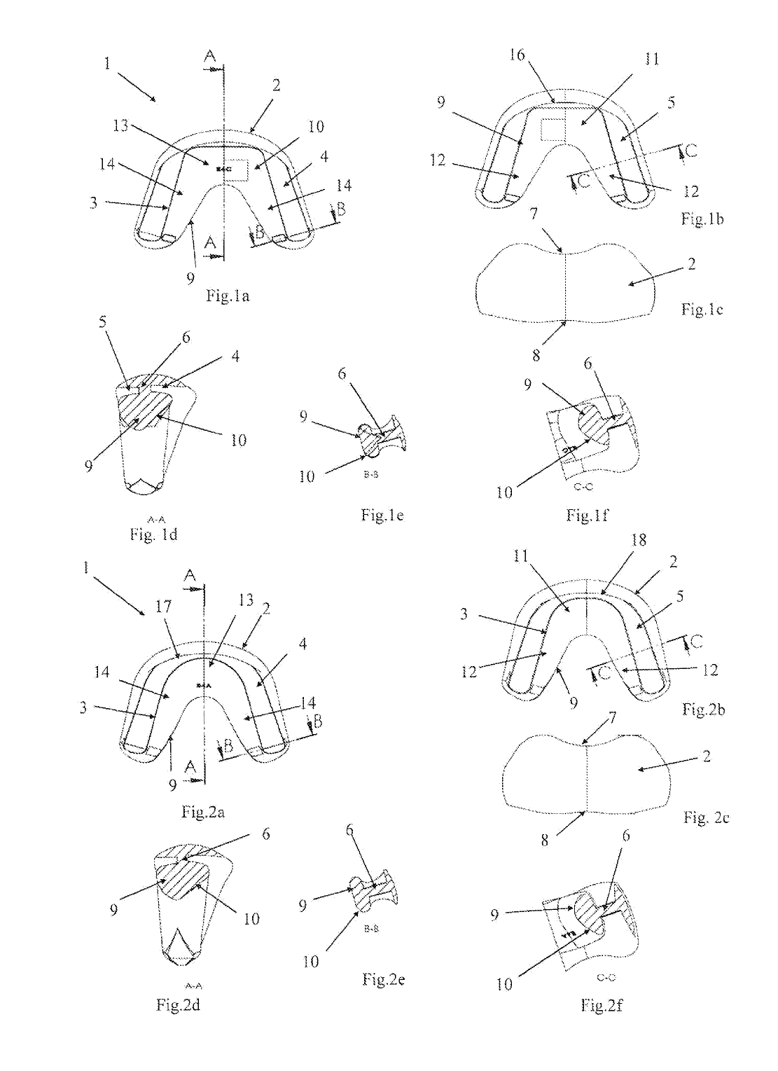

FIGS. 1a to 1f illustrate a first example of an embodiment of the functional dentofacial orthodontic apparatus 1 according to the invention which is a transverse expansion correction apparatus, the top view 1a corresponding to the groove for the upper jaw and the bottom view 1b corresponding to the groove for the lower jaw. The apparatus is of the “splint activator” type, it is a general U shape, as seen in the horizontal plane, it is symmetric with respect to a vertical median plane (represented by the plane A-A in FIG. 1a). The apparatus comprises two vertical bars, one labial bar 2 and one lingual bar 3 defining, with a horizontal partition 6, an upper alveolar canal 4 and a lower alveolar canal 5. The upper alveolar canal 4 and the lower alveolar canal 5 are designed to encompass the maxillary and mandibular arches, respectively, up to the neck. The shape of the central portion of the labial bar 2 can be seen more clearly in FIG. 1c, it consists of a vertical wall having a substantially rectangular shape which reaches the maximum height thereof at the front portion thereof, the height of the labial bar 2 declining at the lateral branches, to the end of the grooves. Two notches are provided at the centre of the bar, one upper 7 for inserting the frenulum of the upper lip and one lower 8 for inserting the frenulum of the lower lip.

In the context of the examples described herein, each apparatus may be embodied in a plurality of sizes. After performing laboratory tests, a set of five or six sizes was selected, according to the model, which proved to be suitable for covering the requirement for apparatuses for children and adults. In this way, for each size, the embodiment of an apparatus according to one of the five or size sizes envisaged is hereinafter understood for each type of example of an embodiment. When adjustments are required, the apparatus having a given size may be suitable for the patient's morphology and/or the defect to be treated by producing notches or attaching material reinforcements in some areas.

More particularly according to the invention, the lingual bar 3 is formed at the end of a reinforced inner raised portion 9 the general shape of which is a flared U, as seen in the horizontal plane, wherein the upper surface forms an angled ramp 10 for supporting and guiding the tongue toward the palate, the cross-section of said raised surface gradually increasing from the end toward the center of the grooves of the apparatus. The reinforced inner raised portion 9 has a central portion 11 and two lateral branches 12. FIGS. 1d, 1e and 1f are cross-sections showing the shape of the grooves and the progression of the shape and dimensions of the reinforced inner raised portion 9 from the end of the lateral branch 12 thereof, seen more clearly in FIG. 1e, in the middle of one of the branches 12 thereof, as represented in FIG. 1f or in the middle of the central portion 11, as seen in FIG. 1d. The reinforced inner raised portion 9 has, as seen in the cross-section, a rounded rim shape at the base thereof, the upper face being beveled, it declines from the upper end of the vertical wall consisting of the lingual bar 3 and forms the angled ramp 10. The angled ramp 10 follows, at the upper part thereof, the contour of the reinforced inner raised relief 9, the surface of the angled ramp 10 defining the bearing surface of the tongue, gradually increasing from the end toward the center of the grooves of the apparatus. The angled ramp 10 is formed from a central slope 13 connected to two lateral slopes 14. The central slope 13 is an angled plane surface formed from the upper edge of the central portion 11, it forms an angle between 30° and 60° with the horizontal, preferably 45°. The lateral slopes 14 are angled plane surfaces each formed from the upper edge of each lateral branch 12 and they form an angle between 30° and 60° with the horizontal, preferably 45°. The angled slopes 13 and 14 are connected using large connection radii so as to obtain a ramp with a smooth surface on the entire periphery thereof.

In the example shown in FIGS. 1a to 1f, the portion of the alveolar canal intended to receive the lower or upper incisors is in the shape of a straight upper 15, or lower 16 canal opening, at both ends thereof, respectively into alveolar canals each intended to receive a lower canine, two lower premolars or a child's two corresponding milk molars and, optionally, at least one lower molar. The length of the straight upper canal 15 exceeds that of the straight lower canal 16 slightly (by approximately 1 to 2 mm). For example, the length of the base of the central portion 11 opening into the straight upper canal 15 is between 28 mm and 37 mm, according to the chosen size of the apparatus.

FIGS. 2a to 2f illustrate a second example of an embodiment of the functional dentofacial orthodontic apparatus 1 according to the invention which is also a transverse expansion correction apparatus. Unlike the apparatus in the first example of an embodiment, in the apparatus shown in FIGS. 2a to 2f, the portion of the alveolar canal intended to receive the lower or upper incisors is in the shape of a rounded upper 17, or lower 18, canal, opening, at both ends thereof, respectively into alveolar canals each intended to receive a lower canine, two lower premolars or a child's two corresponding milk molars and, optionally, at least one lower molar. The length of the rounded upper canal 17 exceeds that of the rounded lower canal 18 slightly (by a few mm). For example, the length of the base of the central portion 11 opening into the rounded upper canal 17 is between 27 mm and 35 mm, according to the chosen size of the apparatus.

The apparatuses produced according to the first and second examples of embodiments are transverse expansion correction apparatuses, the primary action whereof is that of correcting dento-maxillary dysharmony (defects wherein the sizes of the teeth are too large in relation to those of the jaws). Moreover, these apparatuses are envisaged for enabling tooth implantation normalization, satisfactory occlusion and satisfactory functional crossbite. For this, the thickness of the reinforced raised inner portion 9 is very significant on the entire periphery thereof (greater than that of the other correction apparatuses according to the invention, as explained hereinafter) in order to obtain a very strong transverse action. In addition, the apparatus according to the invention enhances nasal ventilation and swallowing considerably, while having a more stimulant action.

For example, the thickness of the inner raised portion measured between the base of the angled ramp 10 and the vertical wall of the lingual bar is between 12.75 mm and 18 mm at the central portion (FIGS. 1d and 2d) and between 3.5 mm and 5.5 mm at the end of the branches (FIGS. 1e and 2e), this thickness varies according to the chosen size of the apparatus, a plurality of sizes being envisaged for children or adults. In the context of these examples, the height of the inner raised portion measured at the central portion is between 18.75 mm and 25 mm.

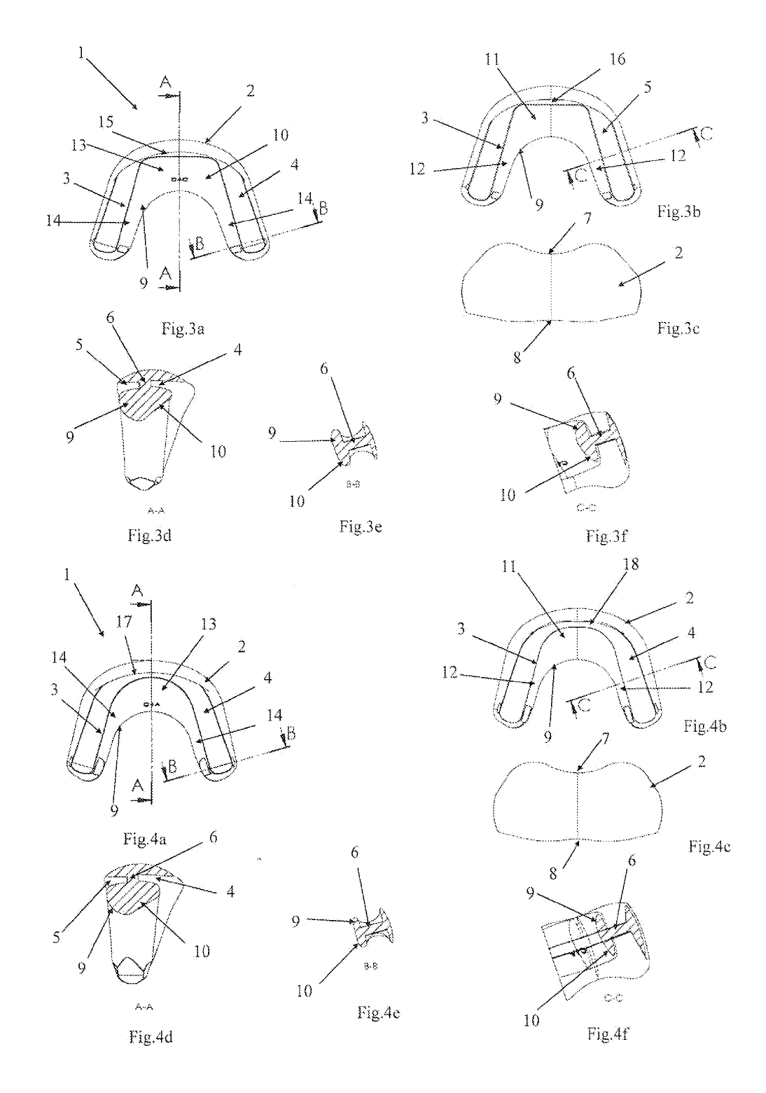

The other examples of apparatuses according to the invention will now be described with reference to FIGS. 3a to 7f, the parts of elements of the apparatuses having the same general shape as those described above have retained the reference numbers thereof. These elements will not be described in further detail hereinafter.

FIGS. 3a to 3f illustrate a third example of an embodiment of the functional dentofacial orthodontic apparatus 1 according to the invention which is a conformer apparatus. A conformer apparatus is used to adjust malpositions of the teeth and vertical dysmorphism, for treating anterior open bite and proalveoli of the upper incisors. A conformer apparatus is particularly used as a retaining apparatus, at the end of the treatment, or when taking a break in the treatment, for example, during a treatment carried out with a transverse expansion apparatus according to any of the examples described above, and the primary function thereof is that of stabilizing the corrections obtained. In the apparatus represented in FIGS. 3a to 3f, the portion of the alveolar canal intended to receive the lower or upper incisors is in the shape of a straight upper 15, or lower 16 canal opening, at both ends thereof, respectively into alveolar canals each intended to receive a lower canine, two lower premolars or a child's two corresponding milk molars and, optionally, at least one lower molar. The length of the straight upper canal 15 is greater than that of the straight lower canal 16. For example, the length of the base of the central portion 11 opening into the straight upper canal 15 is between 29.5 mm and 37 mm, according to the chosen size of the apparatus.

FIGS. 4a to 4f illustrate a fourth example of an embodiment of the functional dentofacial orthodontic apparatus 1 according to the invention which is also a conformer apparatus. Unlike the apparatus in the third example of an embodiment described above, in the example shown in FIGS. 4a to 4f, the portion of the alveolar canal intended to receive the lower or upper incisors is in the shape of a rounded upper 17, or lower 18, canal, opening, at both ends thereof, respectively into alveolar canals each intended to receive a lower canine, two lower premolars or a child's two corresponding milk molars and, optionally, at least one lower molar. For example, the length of the base of the central portion 11 opening into the rounded upper canal 17 is between 27 mm and 35 mm, according to the chosen size of the apparatus.

In the context of the conformer apparatus in FIGS. 3a to 4f, the thickness of the inner raised portion measured between the base of the angled ramp 10 and the vertical wall of the lingual bar is, for example, between 11.25 mm and 15 mm at the central portion (FIGS. 3d and 4d) and between 3 mm and 4 mm at the end of the branches (FIGS. 3e and 4e), this thickness varies according to the chosen size of the apparatus, a plurality of sizes being envisaged for children or adults. In the context of these examples, the height of the inner raised portion measured at the central portion is between 18.75 mm and 25 mm.

By comparing the dimensions of the thickness of the reinforced inner raised portion 9 of the conformer apparatuses illustrated in FIGS. 3a and 4f to those in the transverse expansion apparatuses illustrated in FIGS. 1a to 2f, it is observed that the inner raised portion 9 thereof has larger dimensions, and thus more transverse rigidity than the raised portion of the conformer apparatuses. Consequently, the transverse force applied by a transverse expansion apparatus is greater than that applied by a conformer apparatus which merely stabilizes the effects obtained with the transverse expansion treatment apparatus used.

FIGS. 5a to 5f illustrate a fifth example of an embodiment of the functional dentofacial orthodontic apparatus 1 according to the invention which is an advancement apparatus for retromorphosis or Angle class II which is a mandibular advancement activator, hereinafter referred to as an advancement apparatus. Such an advancement apparatus is generally used for advancing the mandible by sliding forward in the lower portion of the activator to join the ends of the incisors, giving rise to an anatomical adaptation of the arches and elongation of the mandibular base. The advancement apparatus according to the invention has a reinforced inner raised portion further suitable for enhancing nasal ventilation and swallowing while ensuring correct positioning of the tongue on the angled ramp of the inner raised portion, while ensuring a more stimulant action of the appliance. For example, the thickness of the inner raised portion 9 measured between the base of the angled ramp 10 and the vertical wall of the lingual bar 3 is between 13.5 mm and 18 mm at the central portion (figure 5d) and between 4.5 mm and 6 mm at the end of the branches (FIG. 5e), this thickness varies according to the chosen size of the apparatus. In the context of these examples, the height of the inner raised portion measured at the central portion is between 18 mm and 24 mm.

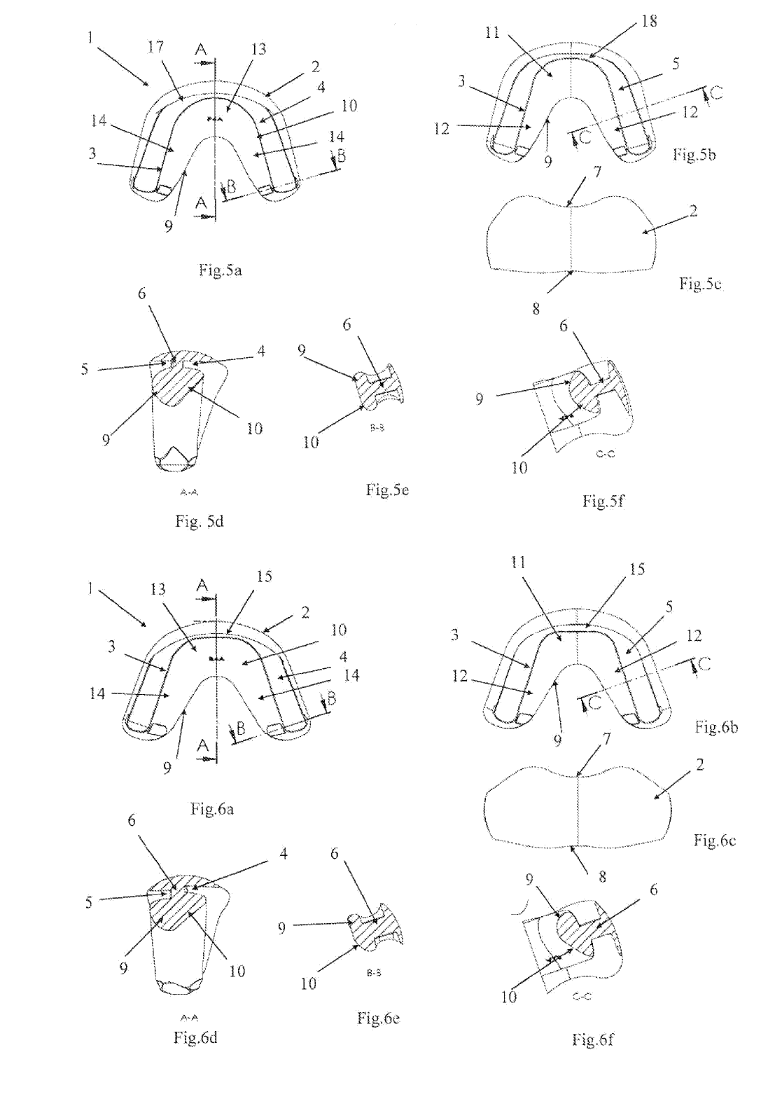

FIGS. 6a to 6f illustrate a sixth example of an embodiment of the functional dentofacial orthodontic apparatus 1 according to the invention which is a retraction device for antemorphosis or Angle class III. The apparatus positions the superior and inferior arches in normal incisor relationships. It has a labial bar 2 having a finer thickness at the upper portion thereof and a lingual bar 3 which is thicker at the upper portion, whereas the thickness of the labial bar at the lower portion thereof is greater. The apparatus applies front to back sagittal pressure, bearing on the superior arch. The retraction apparatus according to the invention has a reinforced inner raised portion further suitable for enhancing nasal ventilation and swallowing while ensuring correct positioning of the tongue on the angled ramp of the inner raised portion, while ensuring a more stimulant action of the appliance. For example, the thickness of the inner raised portion 9 measured between the base of the angled ramp 10 and the vertical wall of the lingual bar 3 is between 10.5 mm and 15 mm at the central portion (FIG. 6d) and between 5.5 mm and 7.5 mm at the end of the branches (FIG. 6e), this thickness varies according to the chosen size of the apparatus. In the context of these examples, the height of the inner raised portion measured at the central portion is between 19.5 mm and 26 mm.

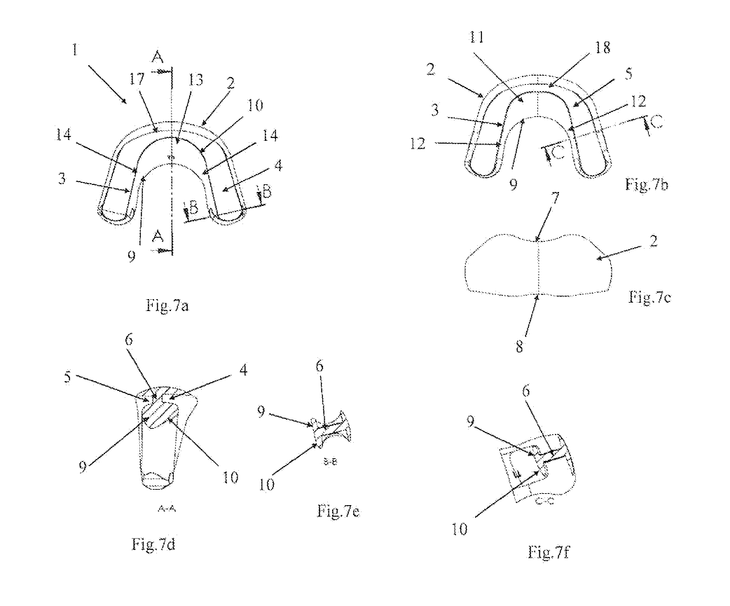

FIGS. 7a to 7f illustrate a seventh example of an embodiment of the functional dentofacial orthodontic apparatus 1 according to the invention which is an apparatus to be used by those practicing sports during training. This apparatus may also be used to stop snoring when used while sleeping, since, due to optimum positioning of the upper and lower jaws, and of the tongue on the reinforced raised portion 10, it ensures satisfactory nasal ventilation reducing snoring. For example, the thickness of the inner raised portion 9 measured between the base of the angled ramp 10 and the vertical wall of the lingual bar 3 is between 10.88 mm and 14.2 mm at the central portion (FIG. 7d) and between 3 mm and 3.5 mm at the end of the branches (FIG. 7e), this thickness varies according to the chosen size of the apparatus. In the context of these examples, the height of the inner raised portion measured at the central portion is between 15.26 mm and 19.23 mm. The dimensions and, consequently, the operating mode of the apparatus in FIGS. 7a to 7f are similar to those of a rounded canal conformer apparatus in FIGS. 4a to 4f. Such an apparatus exerts less force on the arches and is thus more comfortable to use.

The apparatus according to the invention is produced from a silicone base. After performing numerous laboratory tests, it was established that the most suitable material for manufacturing apparatuses according to the invention was STAMINALENE® from STERNE. It consists of a silicone elastomer having enhanced frictional resistance properties and satisfactory endurance, while being very comfortable when worn in the mouth. Such a material has an optimal set of physical characteristics for producing the apparatus according to the invention, i.e.: a hardness of 55 Shore A (as per ASTM D2240), a breaking strength of 10 MPa (as per ASTM D882), a tear strength of 53 kN/m (as per ASTM D624B), an elongation at break of 925% (as per ASTM D412), a compression set of 2.4% measured 30 minutes after the release of the sample from 25% compression for 70 hours. The density thereof is 1.14 g/cm³.

The material is transparent, it may however comprise a dye base using biocompatible dyes at a proportion of 3%. Aromas may also be added to the composition for more pleasant chewing with the apparatus. This silicon comprises a platinum-based catalyst.

In one advantageous embodiment of the invention, the various apparatuses can be distinguished by the color thereof chosen according to the use of the apparatus or the type of defect or neurovegetative function disorder to be treated therewith.

Further examples and embodiments of the invention may be envisaged without leaving the scope of the claims thereof.

1. Functional dentofacial orthopedic apparatus (1) made of a flexible material and comprising two grooves, the general outer shape of each of which is a flared U when viewed horizontally, the two grooves being separated by a substantially horizontal partition (6), each groove defines an alveolar canal (4, 5) encompassing the superior and inferior alveolodental arches, said canal (4, 5) consisting of a labial bar (2) and a lingual bar (3), as well as the horizontal partition (6), the inner surface of each canal being smooth, characterized in that it comprises:
a continuous angled lingual ramp (10) for supporting and guiding the tongue toward the palate formed on the upper portion, said lingual ramp (10) comprising a substantially plane central slope (13) connected to two substantially plane adjacent lateral slopes (14),
a reinforced inner raised portion (9), the general shape of which is a flared U and which extends over the inner periphery of the grooves and the cross-section of which gradually increases from a minimum value at the end of said raised portion to a maximum value in the central portion thereof.

2. Apparatus (1) according to claim 1, characterized in that the depth of each alveolar canal (4, 5) is at least equal to the height of the teeth encompassed therein, to the neck thereof.

3. Apparatus (1) according to claim 2, characterized in that said reinforced inner raised portion (9) has a greater thickness in the area of the alveolar canal of the superior arch and the inferior arch intended to receive the incisors and the canines.

4. Apparatus (1) according to claim 1, characterized in that it is made from food-grade silicon.

5. Apparatus (1) according to claim 4, characterized in that said silicone has a hardness between 50 and 60 Shore, preferably between 53 and 57 Shore.

6. Apparatus (1) according to claim 4, characterized in that said silicone has a compression set between 1.9% and 2.9%, and preferably between 2.2% and 2.6%, measured 30 minutes after the release of the sample from 25% compression for 70 hours.

7. Apparatus (1) according to claim 4, characterized in that the breaking strength of said silicone is between 8 and 12 MPa (as per ASTM D882), and/or the elongation at break of said silicone (measured as per ASTM D412) is between 820 and 1030% and more preferentially between 875 and 975%, and/or the tear strength of said silicone is between 46 and 60 kN/m (as per ASTM D624B), and preferentially between 50 and 56 kN/m.

8. Apparatus (1) according to claim 1, characterized in that the portion of the canal intended to receive the lower or upper incisors is in the shape of a straight canal (15, 16), said canal opens, at both ends thereof, respectively into alveolar canals each intended to receive a lower canine, two lower premolars or a child's two corresponding milk molars and, optionally, at least one lower molar.

9. Apparatus (1) according to claim 1, characterized in that the portion of the canal intended to receive the lower or upper incisors has a rounded canal shape (17, 18), said canal opens, at both ends thereof, respectively into alveolar canals each intended to receive one lower canine, two lower premolars or a child's two corresponding milk molars and, optionally, at least one lower molar.

10. Range of functional dentofacial orthopedic apparatuses, characterized in that it comprises at least two apparatuses according to claim 1 chosen from: a transverse expansion apparatus, a conformer, an advancement apparatus, a retraction apparatus, a sports training apparatus or an anti-snoring apparatus.
